(19) United States
(12) Patent Application Publication
(10) Pub. No.: US 2026/0020872 A1
(43) Pub. Date: Jan. 22, 2026

(54) CUTTING TOOL WITH INTEGRATED RETRACTABLE BLADE SYSTEM FOR INTERATRIAL SHUNT

(71) Applicants: Medtronic CryoCath LP, Toronto (CA); Medtronic, Inc., Minneapolis, MN (US)

(72) Inventors: Nicolas Coulombe, Anjou (CA); Zhongping C. Yang, Woodbury, MN (US); Jorge A. Vergen, Woodbury, MN (US); Lars M. Mattison, Blaine, MN (US)

(21) Appl. No.: 18/854,490

(22) PCT Filed: Mar. 29, 2023
(86) PCT No.: PCT/CA2023/050421

§ 371 Date: Oct. 4, 2024

Related U.S. Application Data
(60) Provisional application No. 63/328,392, filed on Apr. 7, 2022.

(51) Int. Cl. A61B 17/32; A61B 17/00; A61B 90/00

(52) U.S. Cl. CPC A61B 17/32; A61B 17/00234; A61B 90/03; A61B 2017/00252; A61B 2017/00305; A61B 2090/034

(57) ABSTRACT

A method, system, and devices with an integrated retractable blade system for the intraarterial shunt are disclosed. According to one aspect, the cutting tool may comprise an elongate body where the elongate body has a proximal portion and a distal portion opposite the proximal portion. An inner shaft may extend within the distal portion of the elongate body and the inner shaft may have a proximal end and a distal end and a hub may be coupled to the distal end of the inner shaft. The cutting tool may have a cutting element with the hub coupled with the cutting element and movement of the hub transitions the cutting element between a collapsed position and an expanded position.

### FIELD

The present technology is generally related to systems, devices, and methods for creating an interatrial shunt.

### BACKGROUND

Cardiovascular diseases are some of the most common causes of death in the world. Heart failure is one significant cardiovascular disease that is generally known to decrease the heart's ability to pump and/or fill with blood. The number of people diagnosed with heart failure is constantly increasing due to a variety of different issues. In part, this increase may be attributed to more people surviving heart attacks due to scientific advances. Those individuals who have survived a heart attack face a higher heart failure risk afterward as well as challenges related to advancing cardiovascular disease. Also, other health issues are likely major contributors to this rise in the number of people experiencing heart failure. For example, people with diabetes and those that are obese or overweight are at greater risk for developing heart failure and other cardiovascular diseases. Additionally, people with high blood pressure, those who smoke, those with high cholesterol, and people who are generally inactive often have a higher risk for developing heart failure and heart disease. Other risk factors that may contribute to heart failure include an individual's age, gender, and diet. As people live longer and medicine continues to advance, there is more cardiovascular disease and heart failure present within the population.

Elevated pressure within the left atrium of the heart is a known contributor to heart failure and other heart related diseases. Having an elevated pressure within the left atrium can increase the size of the left atrium which has been identified as a precursor of atrial fibrillation, stroke once atrial fibrillation is present, and heart failure.

Health conditions associated with heart failure and left atrial enlargement can cause life altering symptoms or may not be discovered until testing is performed related to other medical issues. Individuals with heart failure may experience a variety of different symptoms that may impact their day-to-day living including, but not limited to, breathlessness or shortness of breath, fatigue, rapid or irregular heartbeats, lack of appetite or nausea, dizziness, swelling, weight gain, chest pain and fainting.

There are limited treatments available to treat a patient with elevated pressures within the left atrium of the heart. While there are preventative measures that can be used to try and lower the risks associated with elevated pressures within the left atrium, these do not cure the problem. For example, eating a healthy diet, exercising regularly, losing weight if overweight, practicing stress management, and managing blood pressure and cholesterol sometimes lessens the effects of these issues which can negatively impact an individual's quality of life. Some available treatments for elevated pressures within the left atrium of the heart include mediations, surgery to repair damaged valves, as well as certain medical devices that may be implanted within the body to help the heart continue to function. Ultimately, there are limited therapies that are available to treat and manage heart failure and elevated pressure within the left atrium of the heart. Heart failure and other cardiovascular diseases often worsen over time and symptoms can become more impactful and limiting on an individual.

Atrial shunting is a procedure used to treat certain cardiac defects and heart failure. During the procedure, a blood flow pathway is created between the right atrium and the left atrium such that blood flows between them. In a typical procedure, the septal wall separating the atria is cut with a puncturing device and a mechanical device such as a stent is left in place to prevent tissue regrowth and to maintain the shunt. Atrial shunting can lessen the symptoms from heart failure and other cardiovascular related diseases.

### SUMMARY

The techniques of this disclosure generally relate to devices and methods for creating an intraarterial shunt to repair and improve symptoms from cardiovascular diseases and to prevent long term-complications related to various cardiovascular diseases.

In one aspect, the present disclosure provides a cutting tool, comprising an elongate body including having a proximal portion, a distal portion opposite the proximal portion. An inner shaft may extend within the distal portion of the elongate body and the inner shaft may have a proximal end and a distal end. A hub may be coupled to the distal end of the inner shaft. The cutting tool may also have a cutting element and the hub may be coupled with cutting element. Movement of the hub may transition the cutting element between a collapsed position and an expanded position.

According to this aspect, in some embodiments, the cutting tool further includes a sheath. The sheath may be movable along the elongate body and sized to cover the cutting element.

According to this aspect, in some embodiments, the hub is rotatably movable within the distal portion via actuation of the inner shaft. Rotation of the hub may transition the cutting element between the collapsed position and the extended position.

According to this aspect, in some embodiments, distal portion of the elongate body includes at least one opening. The cutting element may be sized and configured to be passable through the at least one opening when transitioned between the collapsed position and the extended position.

According to this aspect, in some embodiments, the cutting tool further includes at least one stopper disposed within the distal portion of the elongate body. The at least one stopper may define an aperture sized and configured to receive a portion of one of the inner shaft and the hub and prevent displacement of the hub in a lateral and a vertical direction within the distal portion.

According to this aspect, in some embodiments, the cutting element includes a plurality of blades each having a curved lateral surface. When in the collapsed position, each blade of the plurality of blades at least partially overlaps a first adjacent blade.

According to this aspect, in some embodiments, the plurality of blades are transitionable from the collapsed position to the expanded position when the elongate body is rotated in a first radial direction. The plurality of blades are transitionable from the extended position to the collapsed position when the elongate body is rotated in a second radial direction different from the first radial direction.

According to this aspect, in some embodiments, when in the collapsed position, each blade of the plurality of blades at least partially overlaps with a second adjacent blade.

According to this aspect, in some embodiments, each blade of the plurality of blades is spaced apart from each other when in the extended position.

In one aspect, the present disclosure provides a method of creating a shunt between two chambers of a mammalian heart, including providing a sheath defining a central lumen and a cutting tool. The cutting tool may include an elongate body extending along at least a portion of the central lumen of the sheath, a hub, and a plurality of blades coupled to the hub. The method may further include advancing the cutting tool through the central lumen and positioning the cutting tool proximate to a septal wall, puncturing the septal wall with a distal end of the cutting tool to form an aperture within the septal wall, advancing the cutting tool through the aperture with the plurality of blades in an expanded configuration, and rotating the hub in a first direction to form a shunt within the septal wall.

According to this aspect, in some embodiments, rotating the hub in the first direction transitions the plurality of blades from a collapsed position to an expanded position.

According to this aspect, in some embodiments, rotating the cutting tool in a second direction to transition the plurality of blades from the expanded position to the collapsed position, the second direction being different from the first direction.

According to this aspect, in some embodiments, the method further includes retracting the cutting tool through the central lumen and removing the cutting tool from the sheath.

According to this aspect, in some embodiments, the method further includes providing a medical device having an expandable element, positioning the expandable element at least partially through the aperture, and inflating the expandable element, the expandable element being configured to thermally isolate the septal wall between the left atrium and the right atrium.

According to this aspect, in some embodiments, the method further includes delivering ablation energy to ablate the septal wall.

According to this aspect, in some embodiments, the cutting tool includes a plurality of blades each having a curved lateral surface.

According to this aspect, in some embodiments, when in the collapsed position, each blade of the plurality of blades partially overlaps a first adjacent blade.

According to this aspect, in some embodiments, when in the collapsed position, each blade of the plurality of blades at least partially overlaps a second adjacent blade.

According to this aspect, in some embodiments, each blade of the plurality of blades is spaced apart from an adjacent blade when in the expanded position.

In one embodiment, a method of creating a shunt between two chambers of a mammalian heart, comprising: providing a sheath defining a central lumen and a cutting tool. The cutting tool may include a hub and a plurality of blades coupled to the hub, the plurality of blades each having a curved lateral surface. The method may further include advancing the cutting tool through the central lumen and positioning the cutting tool proximate to a septal wall, puncturing the septal wall with a distal end of the cutting tool to form an aperture within the septal wall, advancing the cutting tool through the aperture when plurality of blades are in a collapsed position, rotating the hub in a first radial direction to transition the plurality of blades from the collapsed position to an expanded position, the transitioning of the plurality of blades to the expanded position cutting the septal wall to form a shunt therein, and rotating the hub in a second radial direction to transition the plurality of blades from the expanded position to the collapsed position, the second radial direction being different from the first radial position.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a first configuration of a catheter and system constructed in accordance with the principles disclosed herein:

FIG. 1A shows a detailed view of a portion of a handle from the catheter of FIG. 1;

FIG. 1B shows an alternate embodiment of an expandable treatment element from the catheter of FIG. 1;

FIG. 2 shows a second configuration of a catheter and system constructed in accordance with the principles disclosed herein;

FIG. 2A shows a detailed view of a portion of the handle from the catheter of FIG. 2;

FIG. 2B shows an alternate embodiment of an expandable treatment element from the catheter of FIG. 1;

FIG. 3A shows a portion of an expandable treatment element of the system of FIG. 1 in a collapsed state;

FIG. 3B shows a portion of an expandable treatment element of the system of FIG. 1 in an expanded state;

FIG. 4A is a view of a cutting element from the expandable treatment element of the system of FIG. 1 in the expanded state;

FIG. 4B is a view of a cutting element from the expandable treatment element of the system of FIG. 1 in the collapsed state;

FIG. 5A shows a portion of an expandable treatment element of the system of FIG. 1 in the expanded state;

FIG. 5B shows a portion of an expandable treatment element of the system of FIG. 1 in the collapsed state;

FIG. 6 shows a portion of an expandable treatment element of the system of FIG. 1 in an expanded state;

FIG. 7 shows a portion of an expandable treatment element of the system of FIG. 1 in an expanded state;

FIG. 8A show a portion of an expandable treatment element of the system of FIG. 1 in the collapsed state;

FIG. 8B show a portion of an expandable treatment element of the system of FIG. 1 in the expanded state;

FIG. 9 is an exemplary method for creating an intraarterial shunt using the expandable treatment element of the system of FIG. 1;

FIG. 10 is an exemplary method for creating an intraarterial shunt using the expandable treatment element of the system of FIG. 1; and

FIG. 11 is an exemplary method for creating an intraarterial shunt using the expandable treatment element of the system of FIG. 1.

### DETAILED DESCRIPTION

Some embodiments include systems and devices for creating an intraarterial shunt within a patient. Some embodiments include using a catheter with at least one cutting element to effectively and efficiently create a shunt within certain body tissue without causing damage to the catheter and to provide control in the location and the depth of creating a shunt within the tissue. In some embodiments, the cutting element may be combined with a form of ablation to create a permanent or more permanent shunt within tissue. These and other features and embodiments are disclosed and discussed in more detail below.

Before describing in detail exemplary embodiments, it is noted that the embodiments reside primarily in combinations of device and system components related to a medical device. Accordingly, the device and system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an”, and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

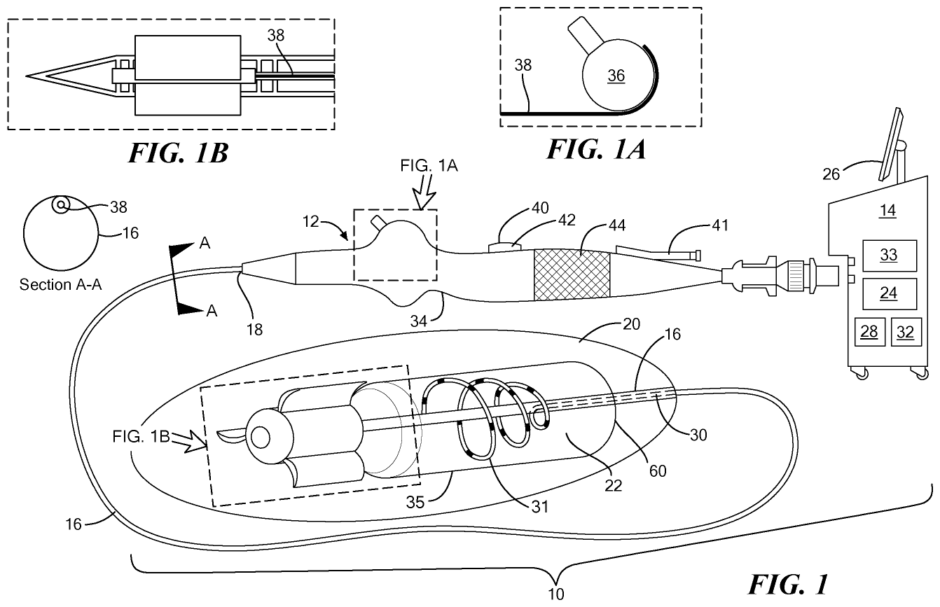

Referring now to the drawing figures where like elements have like reference numerals, FIG. 1 illustrates a medical system with a cutting tool that may be used within the heart and designated generally as “10”. The medical system 10 may include a catheter 12 in electrical and/or fluid communication with a console 14. In one embodiment, the catheter 12 may be a cryoablation catheter, radiofrequency (RF) catheter, or another type of catheter which can be used within the heart of the patient. As shown in FIG. 1, the catheter 12 may have an elongate body 16 that includes a proximal portion 18 and a distal portion 20 opposite the proximal portion 18. Although not shown in FIG. 1, the catheter 12 may be used together with a second catheter such as a guide sheath to assist in positioning the catheter 12 within the heart of the patient or a second catheter with a treatment device that may be used. The elongate body 16 of the catheter 12 is sized and configured to be passable through a patient's vasculature to be positioned proximate to an area within the heart

The catheter 12 may be sized and dimensioned for intraluminal and transseptal access to a patient's heart. The catheter 12 may be formed and dimensioned to provide sufficient column and torsional strength to support standard interventional procedures such as those which access the vasculature from a femoral vein or artery and further access the patient's heart. The elongate body 16 may include reinforcement elements or otherwise be constructed to provide desired degrees of stiffness, flexibility, and torque transmission along the length of the body and at selected locations along its length. The catheter 12 body may have portions or components of differing size, thickness, or flexibility, and may include wires, braiding, changes in wall thickness, additional wall layers or catheter 12 components, sleeves, or other components for reinforcing or otherwise supplementing an outer wall or thickness along its length. Some portions that may experience significant loading or torque during a particular procedure may also include reinforcement. For example, the catheter 12 may be a braided thermoplastic elastomer shaft with different durometer segments to create the desired curve within the catheter 12 depending upon where and how the medical system 10 is going to be maneuvered in the body. The braiding of the thermoplastic elastomer shaft may provide resistance and torque to the catheter 12.

Additionally, the catheter 12 further includes an expandable treatment element 22. It will be understood that the catheter 12 may be used with the expandable treatment element 22 as discussed and other treatment elements may also be used with the catheter 12. As shown in FIG. 1, the expandable treatment element 22 is coupled to, and/or contiguous with, a distal portion 20 of the elongate body 16 so that the expandable treatment element 22 may be passed through the patient's vasculature towards an area of target tissue within the heart, for example. The expandable treatment element 22 may also be flexible to allow for more desirable positioning proximate to an area of tissue. Depending upon the embodiment and configuration of the expandable treatment element 22, the embodiment and configuration may include a tube or sleeve made of memory shape material that is pre-shaped to match the contour of an inner surface of heart for treatment. In some configurations, the expandable treatment element 22 may be a nitinol or polyimide injection tube covered with a thin polymer balloon sleeve. Nitinol is a metal alloy of nickel and titanium and exhibits elasticity and shape memory. More particularly, nitinol has an ability to undergo deformation in response to an applied force at a first temperature, retain the deformed shape at the first temperature when the applied force is removed, and then return to its former shape when heated to a second temperature.

In FIG. 1, the catheter 12 is in electrical and/or fluid communication with the console 14. The console 14 includes one or more controllers, processors, and/or software modules containing instructions or algorithms to provide for the automated operation and performance of the features, sequences, or procedures described herein. In one embodiment, for example, the console 14 includes processing circuitry 24 programmed or programmable to execute the automated or semi-automated operation and performance of the features, sequences, calculations, or procedures described herein. The processing circuitry 24 may include a memory and a processor. In particular, in addition to or instead of a processor, such as a central processing unit, and memory, the processing circuitry 24 may comprise integrated circuitry for processing and/or control, e.g., one or more processors and/or processor cores and/or FPGAs (Field Programmable Gate Array) and/or ASICs (Application Specific Integrated Circuitry) adapted to execute instructions. The processing circuitry 24 may be configured to access (e.g., write to and/or read from) the memory, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory. The memory is in electrical communication with the processor and includes instructions that, when executed by the processor, configure the processor to receive, process, or otherwise use signals from the catheter 12 and/or other system components. Still further, the console 14 may include one or more user input devices, controllers, speakers, and/or displays 26 for collection and conveying information from and to the user.

As shown in FIG. 1, the console 14 further includes a fluid supply reservoir 28 containing cooling fluid. In certain embodiments, the catheter 12 includes a flexible fluid supply lumen 30 extending through a lumen defined by the elongate body 16 and within the expandable treatment element 22. The flexible fluid supply lumen 30 is in fluid communication with the fluid supply reservoir 28 and/or console 14. The processing circuitry 24 is configured and/or programmed to initiate a delivery of cooling fluid from the fluid supply reservoir 28 to the catheter 12 so that the expandable treatment element 22 may be expanded and cooled to remove heat from the tissue to be ablated. Following the completion of an ablation procedure or other cryogenic procedure, the dispersed cooling fluid may pass from the expandable treatment element 22, through the lumen of the elongate body 16 towards the proximal portion 18, and to a fluid recovery reservoir 32 and/or scavenging system.

Further, the fluid recovery reservoir 32 and/or scavenging system referenced to herein may be physically located within or external to the console 14 (as shown in FIG. 1). In one configuration, the console 14 includes the fluid supply reservoir 28, the fluid recovery reservoir 32 for recovering or venting expended cooling fluid for re-use or disposal, and various control mechanisms. In addition to providing an exhaust function for the fluid supply, the console 14 may also include pumps, valves, controllers, or the like to recover and/or re-circulate cooling fluid delivered to the elongate body 16 and/or the fluid pathways of the system 10. Further, the console 14 may include a vacuum pump for creating a low-pressure environment in one or more conduits within the catheter 12 so that refrigerant/coolant fluid is drawn into the conduit(s)/lumen(s) of the elongate body 16. However, as mentioned above, the fluid supply lumen 30, the fluid recovery reservoir 32, or scavenging system may instead be separate from, but in communication with, the console 14.

The medical system 10 may also be configured to deliver radiofrequency energy, such as microwave energy, to tissue, such as the atrium septum to create an intraarterial shunt and may be coupled with an energy source 33 to deliver the energy to the catheter 12 as well. The medical system 10 may be in communication with, for example, a radiofrequency generator 33 that is configured to deliver energy to the catheter 12. The elongate body 16 may have electrical wires that are in communication with the expandable treatment element 22 so that energy may be delivered from the energy source 33 to expandable treatment element 22. Additionally, or alternatively, a secondary treatment device 31, as shown in FIG. 1, may be used with the catheter 12 and the secondary treatment device 31 may be a device that has cryoablation capabilities such as a balloon with electrodes or another such as a helical coil with electrodes. The electrodes on the secondary treatment device 31 may be in communication with the energy source 33 and energy may be delivered from the energy source 33 to the secondary treatment device 31 so that when the secondary treatment device 31 is near tissue, the energy is delivered to tissue. It will be understood that any reference to energy in this application being delivered by the medical system 10 may include, but is not limited to, electromagnetic energy, microwave energy, radiofrequency energy, as well as pulsed field ablation energy/irreversible electroporation energy. The secondary treatment device 31 may optionally have a first sheath 35 that may be placed over or contain the secondary treatment device 31 as the secondary treatment device 31 is maneuvered through the body. Having the first sheath 35 may allow the secondary treatment device 31 to be contained within the first sheath 35 and create a smooth surface so that tissue is not injured. Alternatively, the secondary treatment device 31 may be retained entirely within the elongate body 16 while being maneuvered through tissue and the secondary treatment device 31 may be deployed outside the elongate body 16 before treatment is to be delivered to tissue.

Now referring to FIGS. 1 and 1A, the catheter 12 may also include a handle 34 and the handle 34 may have variety of different mechanical components in communication with the expandable treatment element 22. These mechanical components may include a mechanical switch 36 to which a push wire 38 is connected. It will be understood that the push wire 38 may be used in conjunction or separately from a pull wire as well for steering the catheter. As shown in FIG. 1B, the expandable treatment element 22 may be configured with the push wire 38 that is internal to the expandable treatment element 22 and the push wire 38 may extend from the mechanical switch 36 through the elongate body 16 to the expandable treatment element 22. As shown in Section A-A in FIG. 1, the push wire 38 may be in a lumen inside the elongate body 16 and extend from the handle 34 to the distal portion 20. FIG. 1B shows an alternate embodiment of the expandable treatment element 22 as shown in FIG. 1 on the distal portion 20 with the push wire 38 in communication with the expandable treatment element 22. It will be understood that there are a number of different embodiments of the expandable treatment element 22 that may be used with the system 10. As shown in FIG. 1B, the push wire 38 may be secured to a portion of the expandable treatment element 22 and the push wire 38 may be configured to work in a variety of different manners. For example, when the switch 36 is moved in a particular direction, a portion of the catheter 12, including the distal portion 20, can be moved to the left, right, and/or up or down based upon the corresponding movement of the push wire 38 from the movement of the switch 36. For example, the switch 36 may be movable in a forward direction and a rearward direction. Movement of the switch 36 in the forward direction can be used to move the distal portion 20 left and right. The forward direction may have movement to a first position which moves the distal portion 20 to the left with the push wire 38 and the forward direction may have movement to a second position which moves the distal portion to the right with the push wire 38. Movement of the switch in the rearward direction can be moved to move the distal portion 20 up and down by moving the push wire 38. The rearward direction may have movement to a first position which moves the distal portion 20 upward and the rearward direction may be moved to a second position which moves the distal portion 20 in a downward direction through the movement of the push wire 38.

The handle 34 may also include one port 41 or more than one port 41. As a non-limiting example, the one port 41 may be configured to receive a device which may include a puncturing device and/or a guidewire. In one example, the catheter 12 may be sized to receive the puncturing device, the guidewire, and/or any other medical device that may be inserted into the port 41. Additionally, a different port 41 or the same port 41 may be configured to receive an inflation and deflation lumen as well as other devices that may be used with the medical system 10. The handle 34 may also have additional ports 41 and is not limited to one or two ports 41 and the port 41 may be configured to receive a variety of different devices depending upon how and where the medical system 10 is being used.

The handle 34 may further include an actuator 40 in communication with the expandable treatment element 22 and configured to move the expandable treatment element 22 between a collapse configuration 56 and an expanded configuration 58. The actuator 40 may have a moveable component 42 that can be moved in various directions including up, down, forward, and backward. For example, the movable component 42 may be a bar which is secured to the actuator 40 that can be moved up and down as well as in a forward and rearward direction. If the movable component 42 includes a button, depressing the button may move the expandable treatment element 22 into the expanded configuration 58 and releasing the button may move the expandable treatment element 22 into the collapsed configuration 56. Also, the movable component 42 may be pushed down or depressed and pushing down/depressing the movable component 42 may lock the movable component 42 into place so that the actuator 40 cannot be moved or the movable component 42 can be moved in an upward direction and this movement may release the locking mechanism so that the actuator 40 can be freely movable.

The handle 34 may include one or more steering or deflection components for manipulating the catheter 12, the distal portion 20, and/or any additional components of the medical system 10. In the embodiment of the handle shown in FIG. 1, the handle 34 has a gripping element 44 which gives the user of the medical device a more secure way to hold the handle 34 in one hand while also being able to move the movable component 42 of the actuator 40 in a variety of different directions. For example, the gripping element 44 may be part of the handle 34 to allow the user of the medical system 10 to provide a more secure way to wrap the hand around the handle 34 and the gripping element 44 may be releasably or permanently secured to the handle 34. As shown in FIG. 1, the gripping element 44 is integrated within the body of the handle 34 but the gripping element 44 may be releasably secured to the handle 34 so that a customized gripping element 44 may be used with the handle 34. The gripping element 44 may be ergonomically profiled on one portion of the handle 34 so that it conforms to the user's hand shape. Additionally, the gripping element 44 may have friction-increasing material to help with stability when using the medical system 10 with one or two hands. The friction increasing material may include an elastic rubber material, a coating, a friction increasing strip or another material that may increase the friction between the hand of the user and the gripping element 44 of the handle 34.

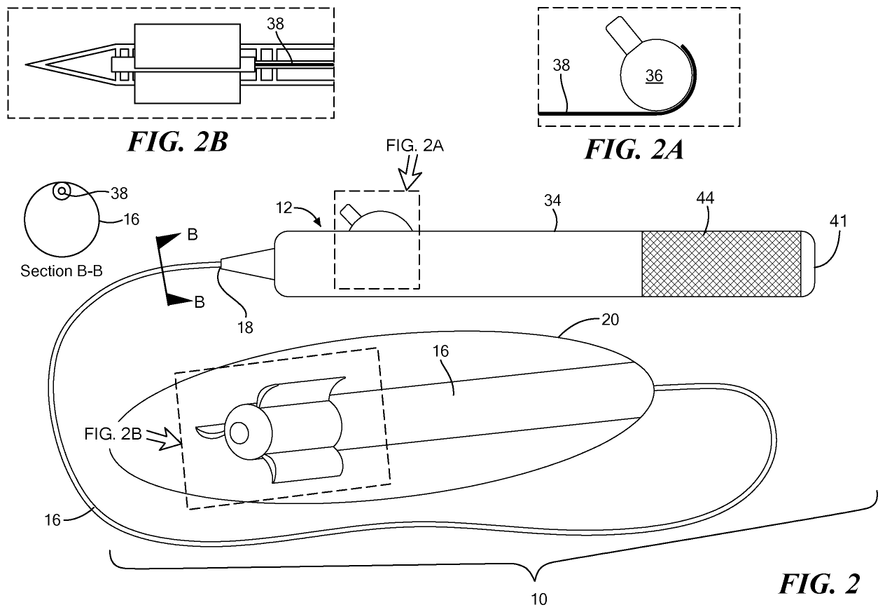

Referring now to FIG. 2 is another embodiment of the medical system 10 without the cryoablation. Cryoablation may be used with the expandable treatment element 22 but in certain situations cryoablation may not be available and/or may not be used with the medical system 10. The medical system 10 may have the switch 36 which is in communication with the push wire 38 and the push wire 38 may extend from the switch 36 to a portion of the expandable treatment element 22. Movement of the switch 36 in different directions may cause the movement of the expandable treatment element 22 in different directions including left, right, up and down. This type of movement can allow the expandable treatment element 22 to be maneuvered within tissue and navigated into specific locations within the tissue. For example, the expandable treatment element 22 may be inserted into tissue and used to cut and/or treat certain tissue and then the expandable treatment element may then be removed from the tissue.

Now referring to FIGS. 2 and 2A, the catheter 12 may also include the handle 34 and the handle 34 may have variety of different mechanical components in communication with the expandable treatment element 22. These mechanical components may include the mechanical switch 36 to which a push wire 38 is connected. As shown in FIG. 2B, the expandable treatment element 22 may be configured with the push wire 38 that is internal to the expandable treatment element 22 and the push wire 38 may extend from the mechanical switch 36 through the elongate body 16 to the expandable treatment element 22. As shown in Section B-B in FIG. 2, the push wire 38 may be in a lumen inside the elongate body 16 and extend from the handle 34 to the distal portion 20. FIG. 2B shows an alternate embodiment of the expandable treatment element 22 as shown in FIG. 2 on the distal portion 20 with the push wire 38 in communication with the expandable treatment element 22. It will be understood that there are a number of different embodiments of the expandable treatment element 22 that may be used with the system 10.

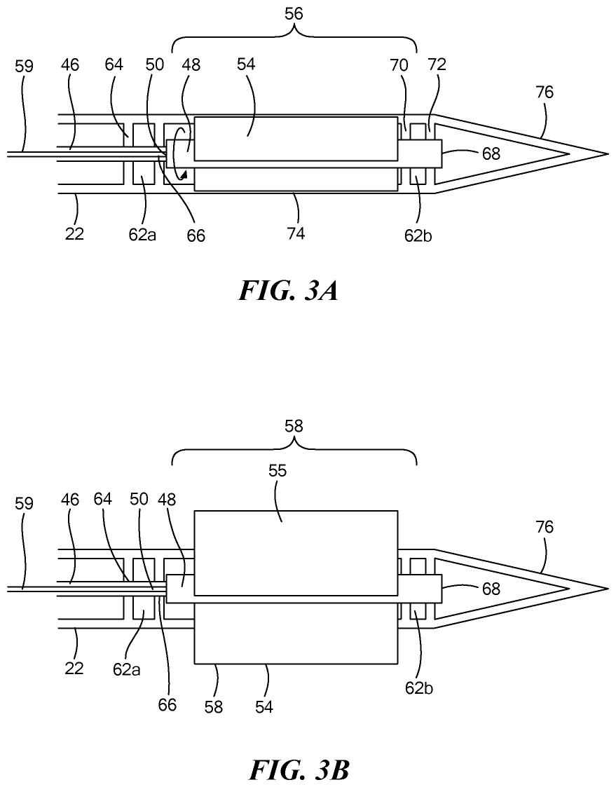

Referring now to FIGS. 3A and 32B is the expandable treatment element 22 in an expanded and collapsed configuration. The expandable treatment element 22 may include an inner shaft 46 and the inner shaft 46 may be in communication with the actuator 40 and/or the switch 36. The inner shaft 46 may extend within at least a portion of the distal portion 20 of the elongate body 16 and the inner shaft 46 may have a distal end 50 and a proximal end 52. The inner shaft 46 may also be coupled with a hub 48 at the distal end 50 of the inner shaft 46. The body 59 may also be secured to, attached, or otherwise in communication with the hub 48 and the body 59 may extend from the hub 48 through the elongate body 16 to the switch 36 and/or the actuator 40. Additionally, one or more than one cutting elements 54 may be part of the expandable treatment element 22 at each cutting element 54 may have at least one blade 55. Each blade 55 may be sharper than the other portions of the cutting element 54 such that the blade 55 can cut through tissue. The blades 55 may have varying thicknesses and may be made from a metal allow such nickel titanium or another type of metal to allow some flexibility as the blades 55 are expanded outwardly and collapsed within the expandable treatment element 22. The cutting elements 54 may be coupled with the hub 48 and the hub 48 may be rotatably movable within the distal portion 20 of the elongate body 16 by the actuation of the inner shaft 46 using the actuator 40.

The rotation of the hub 48 may transition the cutting elements 54 from a collapsed position to an expanded position. In one example, the inner shaft 46 may be actuated or moved by the actuator 40 and this may rotate the hub 48 in a clockwise or counterclockwise direction and the rotation of the hub 48 in one direction may cause the cutting elements 54 to extend outwardly from the hub 48 and rotation of the hub 48 in another direction may cause the cutting elements 54 to become flush with the hub 48.

When in a retracted or collapsed configuration 56 the cutting elements 54 may be retracted within the hub 48 or may be flush with the hub 48 such that a smooth surface is formed. In the retracted or collapsed configuration 56 the expandable treatment element 22 may be moved throughout the body without puncturing or piercing tissue. In the collapsed configuration 56, each of the cutting elements 54 may overlap one another. The expandable treatment element 22 may be transitioned from the collapsed configuration 56 into the expanded configuration 58 when the cutting elements 54 are going to be used to cut tissue. In the expanded configuration 58 each of the cutting elements 54 may be spaced a distance apart from one another. In the expanded configuration 58, the cutting elements 54 may be square or rectangular shaped. In one embodiment, the gripping element 44 on the handle 34 may be in communication with a body 59 and the body 59 may be connected to the gripping element 44 and sized to fit within the elongate body 16 and extend through the elongate body 16 to the hub 48 on the distal end 50 of the inner shaft 46. The body 59 may be connected to or otherwise in communication with the hub 48 and movement of the gripping element 44 in one direction would move the body 59 in one direction which in turn would move the hub 48 in a clockwise or counterclockwise direction. This movement would move the expandable treatment element 22 from an expanded configuration 58 to a collapsed configuration 56 depending upon the direction of movement.

The cutting elements 54 on the expandable treatment element 22 may be other shapes and sizes as well and each cutting element 54 may be the same size, each cutting element 54 may be different sizes, or some of the cutting elements 54 may be the same size and shape while others may be different sizes and shapes. The distal portion 20 of the elongate body 16 of the catheter 12 may include at least one opening 60 and the expandable treatment element 22 may be movable through the elongate body 16 and may be passable through at least one opening 60 of the elongate body 16. When the expandable treatment element 22 is in the collapsed configuration 56, the expandable treatment element 22 may be sized to be passable through the at least one opening 60.

The expandable treatment element 22 may also include at least one stopper 62. The at least one stopper 62 in one exemplary embodiment as shown in FIGS. 2A and 2B may be two stoppers 62. A first stopper 62a may be disposed near the distal end 50 of the inner shaft and in the distal portion 20 of the elongate body 16. The first stopper 62A may define a first aperture 64 and the first aperture 64 may be sized to receive at least a portion of the inner shaft 46 and a second aperture 66 have a first portion sized to receive at least a portion of the inner shaft 46 and a second portion sized to receive at least a portion of the hub 48. The first and second apertures 64 and 66 may be sized to securely hold the hub 48 to prevent displacement of the hub 48 in a lateral or vertical direction. A second stopper 62b may be disposed near a distal end 68 of the hub 48 and the second stopper 62b may have a first aperture 70 and a second aperture 72 sized to receive the hub 48 and the hub may be passable through the first aperture 70 to the second aperture 72 while the distal end 68 of the hub 48 may protrude outwardly from the second stopper 62b. The expandable treatment element 22 may also include a dilator 74 and the dilator 74 may be sized and shaped to hold the cutting elements 54 in place and ensure stable rotation of the hub 48 to allow the cutting elements 54 to be retained within the dilator 74 or extended outwardly from the dilator 74 in an expanded configuration 58. The dilator 74 may also include a dilator tip 76 and the dilator tip 76 may have a traumatic or atraumatic shape. As shown in FIGS. 2A and 2B, the dilator tip 76 may have a traumatic shape and being configured to pierce through tissue within the body so that the expandable treatment element 22 may be passable into certain tissue once the dilator tip 76 is inserted into tissue. The dilator 74 may have each cutting element 54 coupled to a portion of the dilator 74. Having each cutting element 54 coupled with a portion of the dilator 74 will allow for the rotation of the hub 48 so that the cutting elements 54 can move from an expanded to a retracted position while securing the cutting elements 54 in place. Alternatively, the dilator 74 may have a plurality of slits 78 and each slit 78 may be sized and shaped to correspond with a particular cutting element 54. The size and shape of each slit 78 would allow each cutting element 54 to extend outwardly from each slit 78 as well as allowing each cutting element 54 to be retracted within the slit 78 and into the dilator 74. In this configuration, each cutting element 54 may also be secured to the hub 48 so that rotation of the hub 48 in one direction, e.g. clockwise, extends the cutting elements 54 outwardly from each slit 78 and rotation of the hub in another direction, e.g. counterclockwise, retracts each of the cutting elements 54 within each slit 78 so that the cutting elements 54 are flush with the dilator 74.

Now referring to FIGA. 4A and 4B is a view of three cutting elements 54 in an expanded configuration 58 as well as in a collapsed configuration 56 as part of the expandable treatment element 22. In this configuration, each cutting element 54 has a rounded shape and protrudes from the dilator 74. The stopper 62 helps to secure the dilator 74 as well as the cutting elements 54 in place when the expandable treatment element 22 is in the expanded configuration 58. There are three cutting elements 54a, 54b, and 54c which are secured around the rounded dilator 74 and each cutting element 54a, 54b, and 54c has a corresponding blade 55a, 55b, and 55c. When each of the three cutting elements 54a, 54b, and 54c are in the collapsed configuration 56, cutting element 54a is contact with cutting element 54b, cutting element 54b is in contact with cutting element 54c, and cutting element 54c is in contact with cutting element 54a. Each of the cutting elements 54 may be collapsed in a variety of different configurations where each cutting element 54 is in contact with at least one other cutting element.

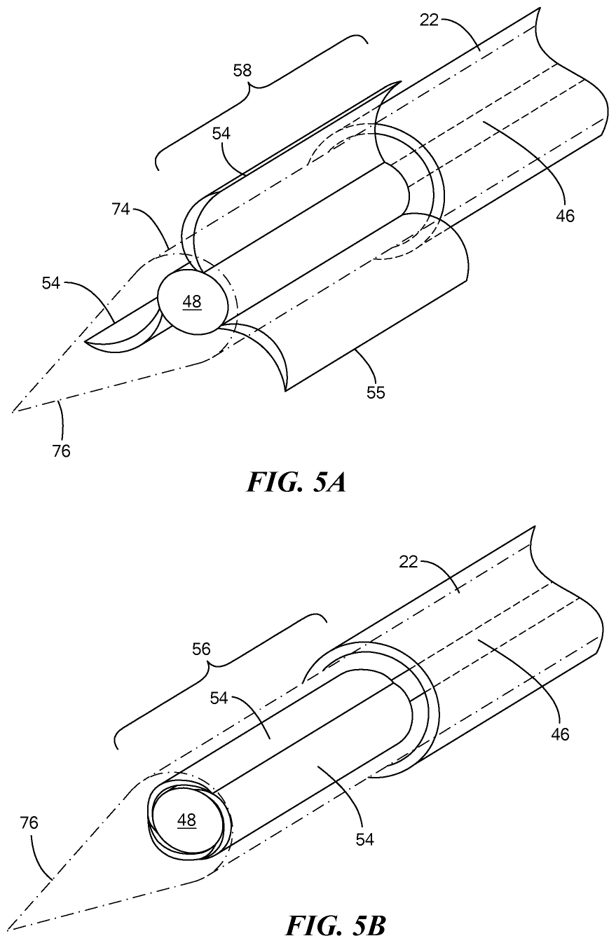

Now referring to FIGS. 5A and 5B, another embodiment of the expandable treatment element 22 is shown in an expanded and a collapsed configuration 56 and 58. Each cutting element 54 has a curved shape and this curved shape on the lateral surface of each cutting element 54 allows the cutting elements 54 as well as the blades 55 to be flush with the dilator 74 when in the collapsed configuration 56 so that the expandable treatment element 22 can be easily moved through the elongate body 16 of the catheter 12 when advancing the retracting the expandable treatment element 22 through the elongate body 16. Each of the cutting elements 54 and each blade 55 may be sized and shape to cut to a predetermined depth as well as a predetermined shape and the sizes and shapes can vary depending upon the tissue, the size the body where the medical system 10 is being used, as well as other factors. The size and shape of the cutting elements 54 and blades 55 may be customized to the particular type of procedure as well as the size, shape, and thickness of the tissue being treated. For example, the blades 55 may be a preset length so that each blade 55 cannot cut past a certain depth. For example, when the expandable treatment element 22 is in the expanded configuration 58, the blades 55 may extend outwardly between 8-10 millimeters and when in the collapsed configuration 56 the blades 55 may be retracted and the expandable treatment element 22 may have a size of between 3-4 millimeters.

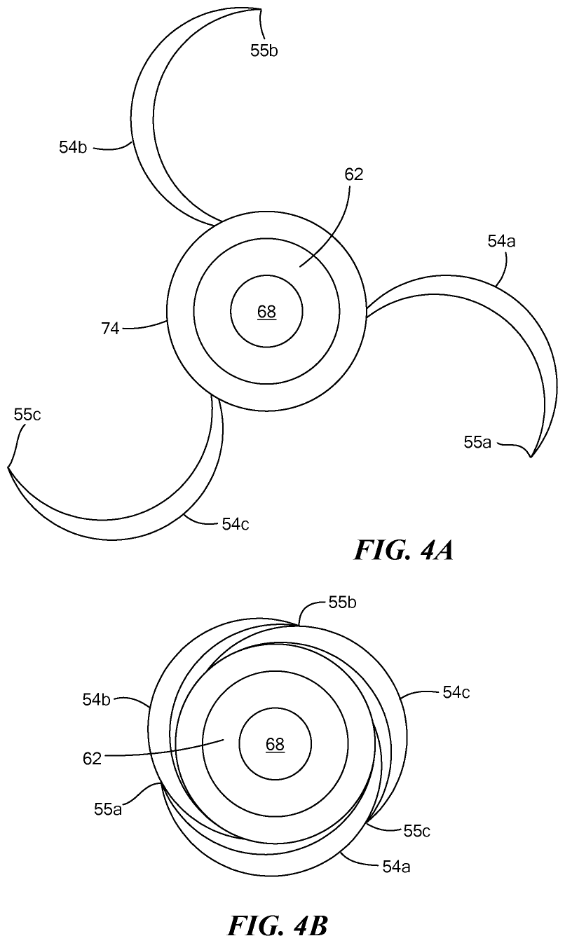

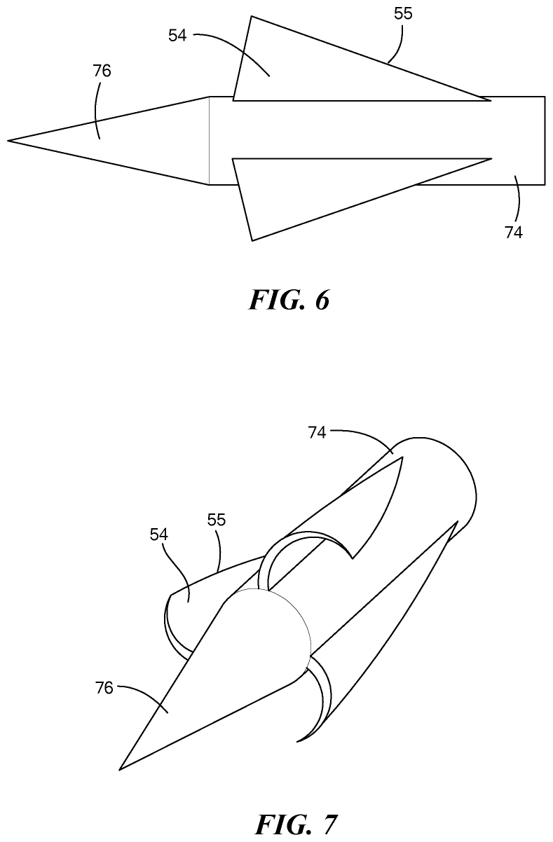

Now referring to FIGS. 6 and 7 are different embodiments of the expandable treatment element 22. In FIG. 4 the cutting elements 54 are shown in an expanded configuration 58 and each of the cutting elements have a tapered blade 55 to facilitate the cutting of tissue as well as the retraction of the tapered blade 55 when the blade 55 is retracted within the dilator 74. The use of the tapered blades 55 may allow for different cutting patterns of tissue as well. The tapered blade 55 may be useful to cut tissue when, for example, the expandable treatment element 22 is moved from the left atrium of the heart to the right atrium. Having the tapered blade 55 when the expandable treatment element 22 is moved from the left atrium to the right atrium of the heart can help avoid the tapered blade 55 from getting caught on tissue and/or tearing or damaging the expandable treatment element 22.

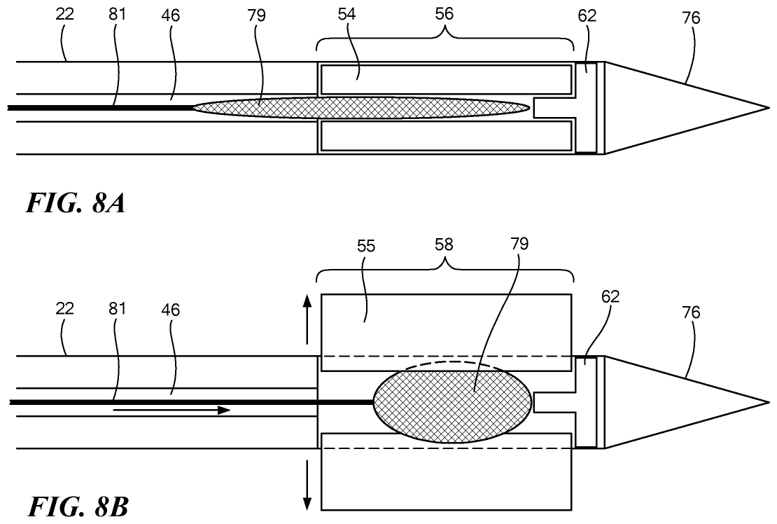

Now referring to FIGS. 8A and 8B are two embodiments of the expandable treatment element 22 wherein the at least one blade 55 is in communication with the inner shaft 46. A movable element 79 may be retained within the inner shaft 46 and the movable element 79 may be transitionable between a variety of different configurations. The movable element 79 may be made out of a variety of different materials including nickel titanium and the movable element 79 may be a mesh or alternatively another malleable material that can be moved into different configurations. A second push wire 81 may be secured to the movable element 79 and the second push wire 81 may extend from the expandable treatment element 22 through the elongate body 16 to the actuator 40 and the movable component 42, the switch 36, and/or the gripping element 44. In one exemplary embodiment, as shown in FIG. 2, the second push wire 81 may be in communication with the gripping element 44 and the gripping element 44 may be rotated in a clockwise or counterclockwise direction and this rotation can cause the movable element 79 to change configurations. For example, as shown in FIG. 8A, the rotation of the gripping element in the clockwise direction may elongate the movable element 79 such that the movable element 79 is not in contact with the at least one cutting element 54 and the expandable treatment element 22 is in the collapsed configuration 56. The turning of the gripping element in the clockwise direction may move the second push wire 81 into a first position where the movable element 79 is pulled into a first configuration where the movable element 79 is expanded. The gripping element 44 may be moved in a counterclockwise direction which moves the second push wire 81 toward the dilator tip 76 and the moveable element 79 may be moved into a second configuration where the movable element 79 is compressed and exerts a force on the at least one cutting element 54. The compression of the movable element can move the at least one cutting element 54 from the collapsed configuration 56 to the expanded configuration 58. Alternatively, the movement of the movable component 42 of the actuator 40 and the corresponding response to the second push wire 81 based upon the position of the movable component 42 of the actuator 40 can move the expandable treatment element from the collapsed configuration 56 to the expanded configuration 58.

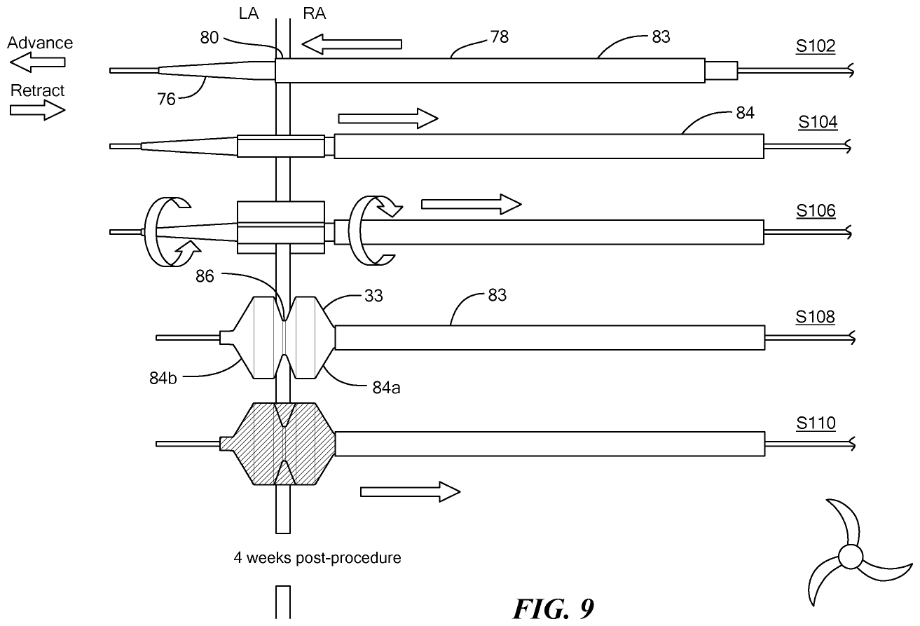

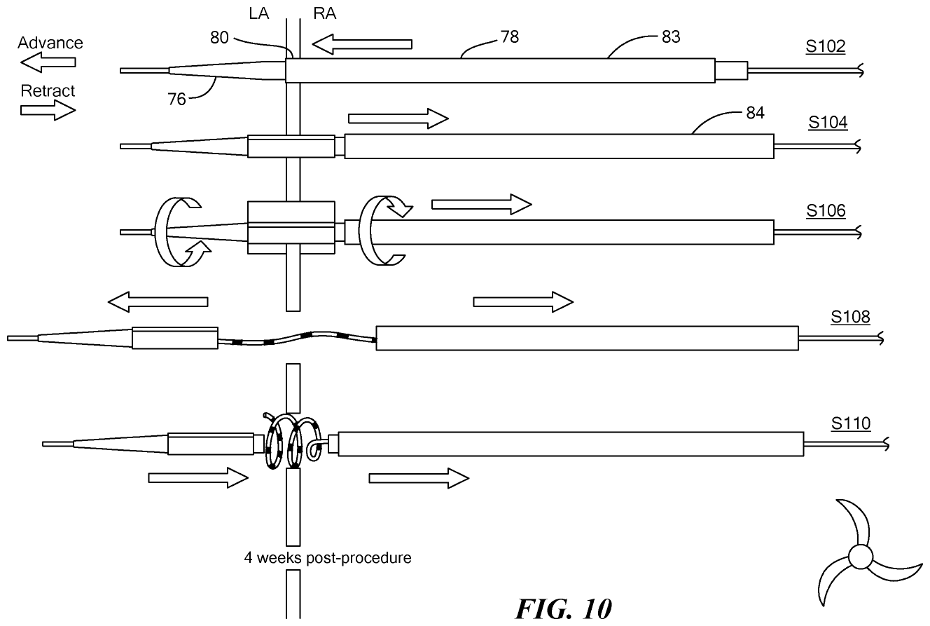

Referring now to FIGS. 9 and 10, is a method of creating an intraarterial shunt includes puncturing of the atrial septum between the right atrium (“RA”) and the left atrium (“LA”) to create a shunt. It will be understood that these methods discuss the atrial septum, but it will be understood that any intraarterial locations may be used for the procedure. For, example, the method includes puncturing a septal wall between a right atrium and a left atrium with the catheter 12 to create an opening in the septal wall. S102. The catheter 12 may be advanced, for example, through the femoral vein to access the right atrium. The medical system 10, according to one embodiment of the disclosure, may be configured to be maneuvered through a patient's vascular system, or another portion of a patient's body. As a non-limiting example, the medical system 10 may be navigated into the left atrium of the heart through the femoral vein and then into the right atrium of the heart via the interior vena cava. Once the medical system 10 is navigated into the right atrium of the heart, the medical system 10 may be inserted into the left atrium of the heart via a transseptal puncture. Once the medical system 10 is in the left atrium, the medical system 10 may be used to puncture through the atrial septum from the left atrium of the heart into the right atrium of the heart, such as through the area of septal tissue known as the fossa ovalis. The medical system 10 may also be maneuvered through other areas of the body as well. The catheter 12 may include the expandable treatment element 22 in the collapsed configuration 56 with the dilator tip 76 in communication with a source of radiofrequency (“RF”) energy (not shown) such that the dilator tip 76 may be configured to pace the right atrium to locate the Fossa Ovalis for a target area to create an opening 80 in the septal wall. For example, the dilator tip 76 may use unipolar pacing RF energy to acquire electrogram signals to locate the Fossa Ovalis. When the Fossa Ovalis is located, the same dilator tip 76 may then use ablative RF energy to puncture the septal wall to create the opening 80. Alternatively, in one configuration, the dilator tip 76 and the dilator 74 may be used to expand the opening 80 by puncturing the tissue with the dilator tip 76. The dilator tip 76 may be advanced through the opening 80 from the right atrium to the left atrium and the dilator 74 may also be advanced through the opening 80 from the right atrium to the left atrium to expand the opening 80.

When the expandable treatment element 22 is advanced into the opening 80, the dilator 74 from the expandable treatment element 22 may be retained within the opening 80. S104 The expandable treatment element 22 may be in the collapsed configuration 56 when inserted into the opening 80. For example, the dilator 74 may be advanced into the opening in the collapsed configuration 56 so that the cutting element 54 is within the opening but the blades 55 are not in contact with the tissue. In the embodiments as shown in FIGS. 9 and 10, the expandable treatment element 22 may include a sheath 83 and the sheath 83 may be movable to cover the cutting element 54 and dilator 74 on the expandable treatment element 22. It will be understood that the sheath 83 may be the same as the first sheath 35 or it may be a different sheath. When the expandable treatment element 22 is moved through tissue within the body the sheath 83 may be used to smoothly maneuver the expandable treatment element 22 through the tissue without damaging tissue. Once the expandable treatment element 22 is within the opening 80, the sheath 83 may be retracted and the cutting element 54 may be exposed. When the sheath 83 is retracted the expandable treatment element 22 may still be in the collapsed configuration 56 and when the expandable treatment element 22 is within the sheath 83, the expandable treatment element 22 may be in the collapsed configuration 56.

When the expandable treatment element 22 is within the opening 80, the expandable treatment element may be transitioned from the collapsed configuration 56 to the expanded configuration 58. S106 The transition from the collapsed configuration 56 to the expanded configuration may be accomplished using the actuator 40 and movable component 42 on the handle 34, the switch 36 and/or the gripping element 44. For example, the actuator 40, the switch 36, and/or the gripping element 44 may be in communication with the expandable treatment element 22 and movement of at least one of these components on the handle 34 may transition the expandable treatment element from the collapsed configuration 56 to the expanded configuration 58. When the expandable treatment element 22 is in the expanded configuration 58, the cutting elements 54 may be used to cut the tissue around the opening 80. The cutting elements 54 may be turned in one direction (e.g., clockwise) to cut the tissue. The cutting pattern of the cutting elements 54 will depend upon how the cutting elements 54 are oriented on the expandable treatment element 22, how many cutting elements 54 are used, and the size of the cutting elements 54. Once the tissue in the opening 80 is sufficiently cut by the cutting elements 54, the expandable treatment element 22 may be transitioned from the expanded configuration 58 to the collapsed configuration 56. In one configuration, the cutting elements 54 may be transitioned from the expanded configuration 58 to the collapsed configuration 56 and the sheath 83 may be placed over the dilator 74 to create a smooth surface so that cutting element 54 will not cut further tissue. Alternatively, the cutting elements 54 may be retained within the dilator 74 or around the dilator 74 so that the surface of the dilator 74 is smooth and the cutting elements 54 are not exposed. The expandable treatment element 22 may be retracted from the opening 80 or the expandable treatment element 22 may be advanced into, for example, the left atrium thereby placing the secondary treatment device 31 in contact with the opening 80.

In the embodiment as shown in FIG. 9, the secondary treatment device 31 may be advanced, for example, over the push wire 38 to a position proximate the opening 80 and then into the opening. S108. As shown in FIG. 9, the secondary treatment device 31 may be inserted into the opening 80. The secondary treatment device 31 may include at least one balloon 84 and the at least one balloon 84 may be advanced at least partially through the opening. In another embodiment as shown in FIG, 10, the secondary treatment device 31 may be a helical wire with electrodes that are disposed near the expandable treatment element 22. S108. The secondary treatment device 31 may be advanced into the opening 80 in an elongated configuration such that the secondary treatment device 31 does not come into contact with the tissue.

As shown in FIG. 9, once the at least one balloon 84 is within the tissue, the at least one balloon 84 may be activated to ablate tissue. S110. The at least one balloon 84 may either be inflated before or after ablation begins. For example, during an RF ablation procedure the balloon 84 may be inflated with saline before RF energy is delivered, or may be inflated with refrigerant, during the ablation procedure. In the configuration as shown in FIG. 9, there are two balloon lobes 84a and 84b and these balloon lobes 84a and 84b are configured to thermally isolate the atrial septum from blood within the left atrium and the right atrium. In this configuration, a middle portion 86 of balloon lobes 84a and 84b are advanced to a position where it is aligned with the atrial septum and the first balloon 84a abuts one side of the septal wall and the second balloon 84b abuts the opposite side of the septal wall. Refrigerant is delivered to the balloon by spraying refrigerant to the middle portion 86 of the balloon 84 to ablate the septal wall that was cut.

Alternatively, as shown in FIG. 10, the helical configuration of the secondary treatment device 31 may be moved from the right atrium of the heart into the opening 80. S110. Once the secondary treatment device 31 is moved into the opening 80, the helical coil may be deformed into a variety of different configurations to come into contact with certain tissue. Once the helical coil is within the opening 80 and in contact with the tissue in the opening, the electrodes on the helical coil may be activated to deliver energy to the tissue.

Once the ablation procedure is completed, the secondary treatment device 31 may be retracted and the shunt remains open without any additional mechanical device being inserted within the shunt to keep the shunt open. The delivery of refrigerant or other energy may prevent regrowth of tissue that was cut and treated with energy during the procedure.

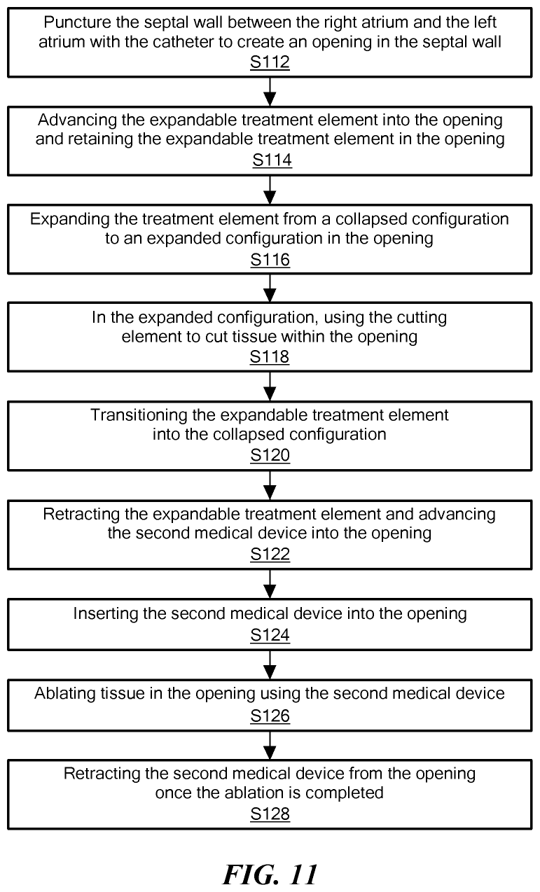

Now referring to FIG. 11 is an exemplary method of treatment creating an intraarterial shunt which includes puncturing of the atrial septum between the right atrium (“RA”) and the left atrium (“LA”) to create a shunt. For, example, the method includes puncturing a septal wall between a right atrium and a left atrium with the catheter 12 to create an opening in the septal wall. S112. The catheter 12 may be advanced, for example, through the femoral vein to access the right atrium. The medical system 10, according to one embodiment of the disclosure, may be configured to be maneuvered through a patient's vascular system, or another portion of a patient's body. As a non-limiting example, the medical system 10 may be navigated into the left atrium of the heart through the femoral vein and then into the right atrium of the heart via the interior vena cava. Once the medical system 10 is navigated into the right atrium of the heart, the medical system 10 may be inserted into the left atrium of the heart via a transseptal puncture. Once the medical system 10 is in the left atrium, the medical system 10 may be used to puncture through the atrial septum from the left atrium of the heart into the right atrium of the heart, such as through the area of septal tissue known as the fossa ovalis. The medical system 10 may also be maneuvered through other areas of the body as well. The catheter 12 may include the expandable treatment element 22 in the collapsed configuration 56 with the dilator tip 76 in communication with a source of radiofrequency (“RF”) energy (not shown) such that the dilator tip 76 may be configured to pace the right atrium to locate the Fossa Ovalis for a target area to create an opening 80 in the septal wall. For example, the dilator tip 76 may use unipolar pacing RF energy to acquire electrogram signals to locate the Fossa Ovalis. When the Fossa Ovalis is located, the same dilator tip 76 may then use ablative RF energy to puncture the septal wall to create the opening 80. Alternatively, in one configuration, the dilator tip 76 and the dilator 74 may be used to expand the opening 80. In particular, the dilator tip 76 may be advanced through the opening 80 from the right atrium to the left atrium and the dilator 74 may also be advanced through the opening 80 from the right atrium to the left atrium to expand the opening 80.

When the expandable treatment element 22 is advanced into the opening 80, the dilator 74 from the expandable treatment element 22 may be retained within the opening 80. S114. The expandable treatment element 22 may be in the collapsed configuration 56 when inserted into the opening 80. For example, the dilator 74 may be advanced into the opening such that the expandable treatment element 22 is in the collapsed configuration 56 so that the cutting element 54 is within the opening but the blades 55 are not in contact with the tissue. The expandable treatment element 22 may include the sheath 83 and the sheath 83 may be movable to cover the cutting element 54 and dilator 74 on the expandable treatment element 22. When the expandable treatment element 22 is moved through tissue within the body the sheath 83 may be used to smoothly move the expandable treatment element 22 through the tissue without damaging tissue. Once the expandable treatment element 22 is within the opening 80, the sheath 83 may be retracted and the cutting element 54 may be exposed. When the sheath 83 is retracted the expandable treatment element 22 may still be in the collapsed configuration 56 and when the expandable treatment element 22 is within the sheath 83, the expandable treatment element 22 may be in the collapsed configuration 56.

When the expandable treatment element 22 is within the opening 80, the expandable treatment element may be transitioned from the collapsed configuration 56 to the expanded configuration 58. S116 The transition from the collapsed configuration 56 to the expanded configuration may be accomplished using the actuator 40 and movable component 42 on the handle 34, the switch 36 and/or the gripping element 44. For example, the actuator 40, the switch 36, and/or the gripping element 44 may be in communication with the expandable treatment element 22 and movement of at least one of these components on the handle 34 may transition the expandable treatment element from the collapsed configuration 56 to the expanded configuration 58. When the expandable treatment element 22 is in the expanded configuration 58, the cutting elements 54 may be used to cut the tissue around the opening 80. S118. The cutting elements 54 may be turned in one direction (e.g., clockwise) to cut the tissue. The cutting pattern of the cutting elements 54 will depend upon how the cutting elements 54 are oriented on the expandable treatment element 22, how many cutting elements 54 are used, and the size of the cutting elements 54. Once the tissue in the opening 80 is sufficiently cut by the cutting elements 54, the expandable treatment element 22 may be transitioned from the expanded configuration 58 to the collapsed configuration 56. S120. In one configuration, the cutting elements 54 may be transitioned from the expanded configuration 58 to the collapsed configuration 56 and the sheath 83 may be placed over the dilator 74 to create a smooth surface so that cutting element 54 will not cut further tissue. Alternatively, the cutting elements 54 may be retained within the dilator 74 or around the dilator 74 so that the surface of the dilator 74 is smooth and the cutting elements 54 are not exposed. The expandable treatment element 22 may be retracted from the opening 80 or the expandable treatment element 22 may be advanced into, for example, the left atrium thereby placing the secondary treatment device 31 in contact with the opening 80.

The expandable treatment element 22 may be retracted from the opening 80 and the secondary treatment device 31 may be advanced, for example, over the push wire 38 to a position proximate the opening 80 and into the opening 80. S122. The secondary treatment device 31 may be inserted into the opening 80. S124 The secondary treatment device 31 may include at least one balloon 84 and/or the helical shaped coil with electrodes and the at least one balloon 84 and/or helical shaped coil with electrodes is advanced at least partially through the opening. For example, the expandable treatment element 22 may have opened the opening 80 and the catheter 12 may be advanced with the secondary treatment device 31 over a guidewire for placement of the at least one balloon 84 and/or the helical shaped coil with electrodes within the opening. The at least one balloon 84 and/or the helical shaped coil with electrodes may ablate tissue once it the balloon is placed within the opening. S126. If the secondary treatment device 31 is the at least one balloon 84, the at least one balloon 84 may either be inflated before or after ablation begins. For example, refrigerant may be delivered to the balloon 84 by spraying refrigerant into the balloon 84 to ablate the septal wall that was cut. Alternatively, energy may be delivered to the electrodes in the helical coil to treatment the tissue that the electrodes is near and/or in contact with. Once the ablation procedure is completed, the secondary treatment device 31 may be retracted and the shunt remains open without any additional mechanical device being inserted within the shunt to keep the shunt open. S128. The delivery of refrigerant may prevent regrowth of tissue that was cut during the procedure.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical system 10.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims and Examples.

Example 1. A cutting tool, comprising: an elongate body including a proximal portion and a distal portion opposite the proximal portion; an inner shaft extending within the distal portion of the elongate body, the inner shaft having a proximal end and a distal end; a hub coupled to the distal end of the inner shaft; and a cutting element, the hub being coupled with the cutting element, wherein movement of the hub transitions the cutting element between a collapsed position and an expanded position.

Example 2. The cutting tool of Example 1, further including a sheath, the sheath being movable along the elongate body and sized to cover the cutting element.

Example 3. The cutting tool of any one of Examples 1-2, wherein the hub is rotatably movable within the distal portion via actuation of the inner shaft, rotation of the hub transitions the cutting element between the collapsed position and the expanded position.

Example 4. The cutting tool of any one of Examples 1-3, wherein: the distal portion of the elongate body includes at least one opening; and the cutting element is sized and configured to be passable through the at least one opening when transitioned between the collapsed position and the expanded position.

Example 5. The cutting tool of any one of Examples 1-4, further including at least one stopper disposed within the distal portion of the elongate body, the at least one stopper defining an aperture sized and configured to: receive a portion of one of the inner shaft and the hub; and prevent displacement of the hub in a lateral and a vertical direction within the distal portion.

Example 6. The cutting tool of any one of Examples 1-5, wherein: the cutting element includes a plurality of blades each having a curved lateral surface; and when in the collapsed position, each blade of the plurality of blades at least partially overlaps a first adjacent blade.

Example 7. The cutting tool of Example 6, wherein: the plurality of blades are transitionable from the collapsed position to the expanded position when the elongate body is rotated in a first radial direction; and the plurality of blades are transitionable from the expanded position to the collapsed position when the elongate body is rotated in a second radial direction different from the first radial direction.

Example 8. The cutting tool of any one of Examples 6-7, wherein, when in the collapsed position, each blade of the plurality of blades at least partially overlaps with a second adjacent blade.

Example 9. The cutting tool of any one of Examples 6-8, wherein each blade of the plurality of blades is spaced apart from each other when in the expanded position.

Example 10. A method of creating a shunt between two chambers of a mammalian heart, comprising: providing a sheath defining a central lumen and a cutting tool, the cutting tool having: an elongate body extending along at least a portion of the central lumen of the sheath; a hub; and a plurality of blades coupled to the hub; and advancing the cutting tool through the central lumen and positioning the cutting tool proximate to a septal wall; puncturing the septal wall with a distal end of the cutting tool to form an aperture within the septal wall; advancing the cutting tool through the aperture with the plurality of blades in an expanded configuration; and rotating the hub in a first direction to form a shunt within the septal wall.

Example 11. The method of Example 10, wherein rotating the hub in the first direction transitions the plurality of blades from a collapsed position to the expanded position.

Example 12. The method of any one of Examples 10-11, further including rotating the cutting tool in a second direction to transition the plurality of blades from the expanded position to the collapsed position, the second direction being different from the first direction.

Example 13. The method of any one of Examples 10-12, further including retracting the cutting tool through the central lumen and removing the cutting tool from the sheath.

Example 14. The method of any one of Examples 10-13, further including: providing a medical device having an expandable element; positioning the expandable element at least partially through the aperture; and inflating the expandable element, the expandable element being configured to thermally isolate the septal wall between the left atrium and the right atrium.

Example 15. The method of any one of Examples 10-14, further including delivering ablation energy to ablate the septal wall.

Example 16. The method of any one of Examples 10-15, wherein the cutting tool includes a plurality of blades each blade having a curved lateral surface.

Example 17. The method of Example 16, wherein, when in the collapsed position, each blade of the plurality of blades partially overlaps a first adjacent blade.

Example 18. The method of Example 17, wherein, when in the collapsed position, each blade of the plurality of blades at least partially overlaps a second adjacent blade.

Example 19. The method of any one of Examples 10-18, wherein each blade of the plurality of blades is spaced apart from an adjacent blade when in the expanded position.

Example 20. A method of creating a shunt between two chambers of a mammalian heart, comprising: providing a sheath defining a central lumen and a cutting tool, the cutting tool having: a hub; and a plurality of blades coupled to the hub, the plurality of blades each having a curved lateral surface; advancing the cutting tool through the central lumen and positioning the cutting tool proximate to a septal wall; puncturing the septal wall with a distal end of the cutting tool to form an aperture within the septal wall; advancing the cutting tool through the aperture so that the plurality of blades is in a collapsed position; rotating the hub in a first radial direction to transition the plurality of blades from a collapsed position to an expanded position, the transitioning of the plurality of blades to the expanded position cutting the septal wall to form a shunt therein; and rotating the hub in a second radial direction to transition the plurality of blades from the expanded position to the collapsed position. the second radial direction being different from the first radial direction.

1. A cutting tool, comprising:
an elongate body including a proximal portion and a distal portion opposite the proximal portion;
an inner shaft extending within the distal portion of the elongate body, the inner shaft having a proximal end and a distal end;
a hub coupled to the distal end of the inner shaft; and
a cutting element, the hub being coupled with the cutting element, wherein movement of the hub transitions the cutting element between a collapsed position and an expanded position.

2. The cutting tool of claim 1, further including a sheath, the sheath being movable along the elongate body and sized to cover the cutting element.

3. The cutting tool of claim 1, wherein the hub is rotatably movable within the distal portion via actuation of the inner shaft, rotation of the hub transitions the cutting element between the collapsed position and the expanded position.

4. The cutting tool of claim 1, wherein:
the distal portion of the elongate body includes at least one opening; and
the cutting element is sized and configured to be passable through the at least one opening when transitioned between the collapsed position and the expanded position.

5. The cutting tool of claim 1, further including at least one stopper disposed within the distal portion of the elongate body, the at least one stopper defining an aperture sized and configured to:
receive a portion of one of the inner shaft and the hub; and
prevent displacement of the hub in a lateral and a vertical direction within the distal portion.

6. The cutting tool of claim 1, wherein:
the cutting element includes a plurality of blades each having a curved lateral surface; and
when in the collapsed position, each blade of the plurality of blades at least partially overlaps a first adjacent blade.

7. The cutting tool of claim 6, wherein:
the plurality of blades are transitionable from the collapsed position to the expanded position when the elongate body is rotated in a first radial direction; and
the plurality of blades are transitionable from the expanded position to the collapsed position when the elongate body is rotated in a second radial direction different from the first radial direction.

8. The cutting tool of claim 6, wherein, when in the collapsed position, each blade of the plurality of blades at least partially overlaps with a second adjacent blade.

9. The cutting tool of claim 6, wherein each blade of the plurality of blades is spaced apart from each other when in the expanded position.

10. A method of creating a shunt between two chambers of a mammalian heart, the method comprising:
providing a sheath defining a central lumen and a cutting tool, the cutting tool having:
an elongate body extending along at least a portion of the central lumen of the sheath;
a hub; and
a plurality of blades coupled to the hub;
advancing the cutting tool through the central lumen and positioning the cutting tool proximate to a septal wall;
puncturing the septal wall with a distal end of the cutting tool to form an aperture within the septal wall;
advancing the cutting tool through the aperture with the plurality of blades in an expanded configuration; and
rotating the hub in a first direction to form a shunt within the septal wall.

11. The method of claim 10, wherein rotating the hub in the first direction transitions the plurality of blades from a collapsed position to the expanded position.

12. The method of claim 10, further comprising:
rotating the cutting tool in a second direction to transition the plurality of blades from the expanded position to the collapsed position, the second direction being different from the first direction.

13. The method of claim 10, further comprising:
retracting the cutting tool through the central lumen and removing the cutting tool from the sheath.

14. The method of claim 10, further comprising
providing a medical device having an expandable element;
positioning the expandable element at least partially through the aperture; and
inflating the expandable element, the expandable element being configured to thermally isolate the septal wall between the left atrium and the right atrium.

15. The method of claim 10, further comprising:
delivering ablation energy to ablate the septal wall.

16. The method of claim 10, wherein the cutting tool includes a plurality of blades each blade having a curved lateral surface.

17. The method of claim 16, wherein, when in the collapsed position, each blade of the plurality of blades partially overlaps a first adjacent blade.

18. The method of claim 17, wherein, when in the collapsed position, each blade of the plurality of blades at least partially overlaps a second adjacent blade.

19. The method of claim 10, wherein each blade of the plurality of blades is spaced apart from an adjacent blade when in the expanded position.

20. A method of creating a shunt between two chambers of a mammalian heart, the method comprising:
providing a sheath defining a central lumen and a cutting tool, the cutting tool having:
a hub; and
a plurality of blades coupled to the hub, the plurality of blades each having a curved lateral surface;
advancing the cutting tool through the central lumen and positioning the cutting tool proximate to a septal wall;
puncturing the septal wall with a distal end of the cutting tool to form an aperture within the septal wall;
advancing the cutting tool through the aperture so that the plurality of blades is in a collapsed position;
rotating the hub in a first radial direction to transition the plurality of blades from a collapsed position to an expanded position, the transitioning of the plurality of blades to the expanded position cutting the septal wall to form a shunt therein; and
rotating the hub in a second radial direction to transition the plurality of blades from the expanded position to the collapsed position, the second radial direction being different from the first radial direction.
